(19) United States
(12) Patent Application Publication
(10) Pub. No.: US 2022/0354834 A1
(43) Pub. Date: Nov. 10, 2022

(54) METHODS AND MATERIALS FOR TREATING NEUROTOXICITY

(71) Applicant: Cavion, Inc., Charlottesville, VA (US)

(72) Inventors: Yuri MARICICH, Charlottesville, VA (US); Evan NEWBOLD, Charottesville, VA (US); Guido CAVALETTI, Milano (IT); Cristina MEREGALLI, Milano (IT)

(21) Appl. No.: 17/766,113

(22) PCT Filed: Oct. 2, 2020
(86) PCT No.: PCT/US2020/053944

§ 371 Date: Apr. 1, 2022

Related U.S. Application Data
(60) Provisional application No. 62/909,694, filed on Oct. 2, 2019.

(51) Int. Cl. A61K 31/4425; A61P 35/00; A61P 25/00; A61K 9/00

(52) U.S. Cl. CPC A61K 31/4425; A61P 35/00; A61P 25/00; A61K 9/0053

(57) ABSTRACT

This document relates to methods and materials for treating a mammal having neurotoxicity (e.g., chemotherapy-induced neurotoxicity). For example, one or more T-type calcium channel modulators (e.g., a composition including one or more T-type calcium channel modulators such as CX-8998) can be administered to a mammal having neurotoxicity to treat the mammal.

### CROSS-REFERENCE TO RELATED APPLICATIONS

This application claims the benefit of U.S. Patent Application Ser. No. 62/909,694, filed on Oct. 2, 2019. The disclosure of the prior application is considered part of (and is incorporated by reference in) the disclosure of this application.

### TECHNICAL FIELD

This document relates to methods and materials for treating a mammal having neurotoxicity (e.g., chemotherapy-induced neurotoxicity). For example, one or more T-type calcium channel modulators (e.g., a composition including one or more T-type calcium channel modulators such as CX-8998 or a metabolite thereof) can be administered to a mammal having neurotoxicity in an amount effective to treat the mammal.

### BACKGROUND

Pivotal multiple myeloma clinical trials have shown that bortezomib (BTZ) alone or in combination therapy provided clinically significant benefits including greater overall response rates, longer time to disease progression and increased overall survival rates (Chen et al., 2011 Curr Cancer Drug Targets 11(3):239-253; Knopf et al., 2014 Clin Lymphoma Myeloma Leuk 14(5):380-388; Aguiar et al., 2017 Crit Rev Oncol Hematol 113:196-212; and Sun et al., 2017 Biosci Rep 37(4):BSR20170304).

However, chemotherapy-induced peripheral neurotoxicity (CIPN) is a serious side effect of chemotherapeutic anti-cancer agents (Argyriou et al., 2012 Crit Rev Oncol Hematol 82:51-77; Cavaletti et al., 2010 Nat Rev Neurol 6:657-666; Grisold et al., 2012 Neurol Oncol 14(Suppl 4):45-54; and Flatters et al., 2017 Br J Anaesth 119:737-749). CIPN has detrimental impacts on motor and sensory neurons and causes nerve fiber degeneration (Carozzi et al., 2013 PLoS One 8:e72995; Cavaletti et al., 2007 Exp Neurol 204:317-325; Gilardini et al., 2012 Neurotoxicol 33:1-7; and Quartu et al., 2014 Biomed Res Int 2014:180428). Within a month of chemotherapy, 68% of cancer patients develop CIPN (Seretny et al., 2014 Pain 155:2461-2470). Current pharmacotherapies do not target CIPN pathophysiologies and cannot reverse the disorder (Starobova et al., 2017 Front Mol Neurosci 10:174; and Hershman et al., 2014 J Clin Oncol 32:1941-1967).

### SUMMARY

Treating and/or preventing CIPN during treatment with chemotherapeutic agents remains an unmet medical need.

This document provides methods and materials for treating a mammal having neurotoxicity (e.g., chemotherapy-induced neurotoxicity), or at risk of developing neurotoxicity (e.g., a mammal scheduled or expected to be administered a chemotherapeutic agent associated with chemotherapy-induced neurotoxicity). For example, one or more (e.g., one, two, three, four, five or more) T-type calcium channel modulators (e.g., CX-8998 or a metabolite thereof) can be administered to a mammal having neurotoxicity to treat the mammal. In some cases, a mammal having neurotoxicity, or at risk of developing neurotoxicity, can be administered one or more T-type calcium channel modulators to treat the mammal.

CX-8998 is a potent and highly selective voltage-activated negative allosteric modulator of T-type calcium channels that can reduce T-type calcium channel activity, and that is safe for use in mammal such as humans (Egan et al., 2013 Hum Psychopharmacol. 28(2):124-133; and Papapetropoulos et al., 2018 Mov Disord. 33(S2):S14 (abstract 29)). As demonstrated herein, co-treatment with (e.g., administration of both) CX-8998 and BTZ did not interfere with BTZ activity on human multiple myeloma cell lines in vitro or on multiple myeloma cell line RPMI-8226 cells in vivo. As demonstrated herein, co-treatment with CX-8998 and BTZ reversed BTZ-induced neurotoxicity. For example, co-treatment with CX-8998 (10 and 30 mg/kg) and BTZ reversed BTZ-induced reduction of nerve conduction velocity (NCV) without interfering with BTZ-induced proteasome inhibition. For example, co-treatment with CX-8998 (30 mg/kg) and BTZ reduced BTZ-induced β-tubulin polymerization and nerve fiber loss. Having the ability to reduce or eliminate neurotoxicity (e.g., chemotherapy-induced neurotoxicity) provides a unique and unrealized opportunity to maximize the therapeutic benefit of a chemotherapy (e.g., the anti-cancer effects) while reducing or eliminating any neurotoxic side effects of the chemotherapy. For example, the ability to reduce or eliminate neurotoxicity (e.g., chemotherapy-induced neurotoxicity) can allow a patient to tolerate a chemotherapy without the need for any dose reduction or termination that could result in inadequate cancer treatment and/or shortened survival.

In general, one aspect of this document features methods for treating a mammal having neurotoxicity. The methods can include, or consist essentially of, administering an effective amount of a composition comprising a T-type calcium channel modulator or a salt thereof to a mammal to reduce a symptom of a neurotoxicity in the mammal. The method also can include identifying the mammal as having a neurotoxicity. The mammal can be a human. The neurotoxicity can be a chemotherapy-induced neurotoxicity. The said chemotherapy-induced neurotoxicity can be a bortezomib-induced neurotoxicity. The mammal having neurotoxicity can have been administered the chemotherapy to treat a cancer within the mammal. The cancer can be multiple myeloma, mantle cell lymphoma, leukemia, digestive tract cancer, lung cancer, testicular cancer, ovarian cancer, brain cancer, uterine cancer, prostate cancer, bone cancer, breast cancer, or bladder cancer. The symptom can be pain, limb weakness, limb numbness, itch, parasthesia, palsy, anosmia, ptosis, chronic cough, motor dysfunction, memory loss, vision loss, headache, cognitive impairment, encephalopathy, dementia, mood disorder, constipation, sexual dysfunction, bladder retention, hemorrhage, or any combinations thereof. The T-type calcium channel modulator can be a negative modulator. The negative modulator can be a negative allosteric modulator. The T-type calcium channel modulator can reduce T-type calcium channel activity. The T-type calcium channel modulator can include CX-8998. The CX-8998 can be in the form of a salt. The T-type calcium channel modulator can include a metabolite of CX-8998. The metabolite of CX-8998 can have a structure of

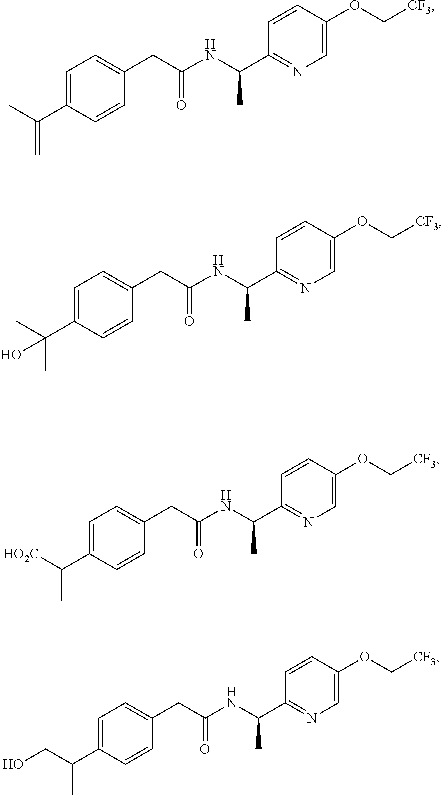 [SMILES: C=C(C)c1ccc(CC(=O)N[C@H](C)c2ccc(OCC(F)(F)F)cn2)cc1 ; C[C@@H](NC(=O)Cc1ccc(C(C)(C)O)cc1)c1ccc(OCC(F)(F)F)cn1 ; CC(C(=O)O)c1ccc(CC(=O)N[C@H](C)c2ccc(OCC(F)(F)F)cn2)cc1 ; CC(CO)c1ccc(CC(=O)N[C@H](C)c2ccc(OCC(F)(F)F)cn2)cc1]

or any combinations thereof. The metabolite of CX-8998 can be in the form of a salt. The T-type calcium channel modulator can include CX-8998 and one or more metabolites of CX-8998. The composition can include from about 10 nM to about 1000 nM of the T-type calcium channel modulator. The composition can include from about 3 mg/kg body weight of the mammal to about 30 mg/kg body weight of the mammal of the T-type calcium channel modulator to the mammal. The composition can be administered orally.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1D. CX-8998 Interference of BTZ Anti-Cancer Activity. (FIG. 1A) Percent survival of multiple myeloma cell lines (MCLs) MM.1S, RPMI8336, and U266B1 treated in vitro for 72 hours with BTZ at the IC₅₀ (6±0.5 nM, 4±1.7 nM and 2.5±0.6 nM respectively) in the presence or absence of various concentrations of CX-8998. (FIG. 1B) Relative body weight in nude mice bearing RPMI8226 xenografts. (FIG. 1C) Tumor volume in nude mice bearing RPMI8226 xenografts. (FIG. 1D) Percent proteasome inhibition in PBMCs isolated from rats.

FIG. 2. Caudal Nerve Conduction Velocity. Conduction velocity obtained from caudal nerves by electromyography during phase 1 (baseline and 4 weeks) and phase 2 (5 and 8 weeks) in a rat model of BTZ-induced CIPN.

FIG. 3. Sciatic Nerve Conduction Velocity. Conduction velocity obtained from sciatic nerves by electromyography during phase 1 (baseline and 4 weeks) and phase 2 (5 and 8 weeks) in a rat model of BTZ-induced CIPN.

FIG. 4. Mechanical Threshold (MT). Evaluation of mechanical allodynia measured using a Dynamic Aesthesiometer Test during phase 1 (baseline and 4 weeks) and phase 2 (5 and 8 weeks) in a rat model of BTZ-induced CIPN.

FIGS. 5A-5C. β-Tubulin Polymerization, Sciatic Nerve Fiber Density and Histopathology. (FIG. 5A) Percent of β-tubulin polymerization in protein extracts from sciatic nerve tissue collected after 8-weeks treatment with BTZ in the presence or absence of CX-8998. (FIG. 5B) Number of nerve fibers per mm quantified in sections of plantar glabrous skin from hind paws collected after 8-weeks treatment with BTZ in the presence or absence of CX-8998. (FIG. 5C) Representative images of tissue samples quantified in FIG. 5B.

### DETAILED DESCRIPTION

This document provides methods and materials for treating a mammal having neurotoxicity (e.g., chemotherapy-induced neurotoxicity), or at risk of developing neurotoxicity (e.g., a mammal scheduled or expected to be administered a chemotherapeutic agent associated with chemotherapy-induced neurotoxicity). For example, one or more (e.g., one, two, three, four, five or more) T-type calcium channel modulators (e.g., CX-8998 or a metabolite thereof) can be administered to a mammal having neurotoxicity to treat the mammal. In some cases, a mammal having neurotoxicity, or at risk of developing neurotoxicity, can be administered one or more T-type calcium channel modulators and/or one or more metabolites thereof to treat the mammal.

In some cases, a mammal (e.g., a human) having, or at risk of developing, neurotoxicity (e.g., chemotherapy-induced neurotoxicity) can be administered one or more (e.g., one, two, three, four, five or more) T-type calcium channel modulators (e.g., CX-8998 or a metabolite thereof) to reduce or eliminate one or more symptoms of neurotoxicity. For example, one or more T-type calcium channel modulators can be administered to a mammal as described herein to reduce the severity of one or more symptoms of neurotoxicity by, for example, 10, 20, 30, 40, 50, 60, 70, 80, 90, 95, or more percent. In some cases, a symptom of neurotoxicity can be a delayed symptom (e.g., can go undetected for hours, day, or weeks after neurotoxicity has been developed). Examples of symptoms of neurotoxicity that can be reduced or eliminated by the methods described herein include, without limitation, pain, weakness (e.g., limb weakness), numbness (e.g., limb numbness), itch, parasthesia, palsy, anosmia, ptosis, chronic cough, motor dysfunction, memory loss, vision loss, headache, cognitive impairment, encephalopathy, dementia, mood disorder, constipation, sexual dysfunction, bladder retention, and/or hemorrhage.

Any appropriate mammal (e.g., a human) having, or at risk of developing, neurotoxicity (e.g., chemotherapy-induced neurotoxicity) can be treated as described herein by administering one or more T-type calcium channel modulators such as CX-8998 or a metabolite thereof. In some cases, a mammal having, or at risk of developing, neurotoxicity can have a disease or disorder that makes the mammal more vulnerable to developing neurotoxicity. Examples of mammals having, or at risk of developing, neurotoxicity that can be treated as described herein include, without limitation, humans, non-human primates such as monkeys, dogs, cats, horses, cows, pigs, sheep, mice, and rats. In some cases, a human having, or at risk of developing, neurotoxicity can be treated by administering one or more T-type calcium channel modulators to the human.

Any appropriate neurotoxicity can be treated as described herein by administering one or more T-type calcium channel modulators such as CX-8998 or a metabolite thereof. Neurotoxicity can affect (e.g., can damage) any appropriate part of the nervous system. In some cases, neurotoxicity can be present in the central nervous system (CNS). In some cases, neurotoxicity can be present in the peripheral nervous system (PNS). In some cases, neurotoxicity can be present in both the CNS and the PNS. Neurotoxicity can cause any type of damage to the nervous system. In some cases, neurotoxicity can include reversible damage to nervous tissue (e.g., to one or more neurons). For example, neurotoxicity can alter the normal activity of the nervous system (e.g., can disrupt the function of one or more neurons). In some cases, neurotoxicity can include permanent damage to nervous tissue (e.g., to one or more neurons). For example, neurotoxicity can kill or impair function of one or more neurons. In some cases, neurotoxicity can be induced by exposure to a particular substance. Causes of neurotoxicity include, without limitation, drug therapies (e.g., chemotherapy), radiation treatment, exposure to heavy metals (e.g., lead and mercury), diabetes, viral infections, nerve injury, hereditary genetic conditions, exposure to pesticides, exposure to solvents (e.g., industrial solvents and cleaning solvents), exposure to molds, foods, food additives, and toxins (e.g., naturally occurring toxins and man-made toxins). In some cases, neurotoxicity can be induced by a chemotherapy (e.g., chemotherapy-induced neurotoxicity). In chemotherapy-induced neurotoxicity, the neurotoxicity can be caused by any chemotherapeutic agent. Examples of chemotherapeutic agents that can cause neurotoxicity when administered to a mammal (e.g., a human) include, without limitation, proteasome inhibitors (e.g., BTZs such as VELCADE®, CHEMOBORT™, and BORTECAD™), epothilones, vinca alkaloids, taxanes, immunomodulatory drugs, anthracyclines, cyclophosphamides, and platinum-based therapies. In chemotherapy-induced neurotoxicity, the chemotherapy can be administered to a mammal (e.g., a human) having any type of cancer. In some cases, a cancer can include one or more solid tumors. In some cases, a cancer can be a hematologic cancer. In some cases, a cancer can be a primary cancer, a metastatic cancer, or a relapsed cancer. Examples of cancers that can be treated with chemotherapeutic agents that can cause chemotherapy-induced neurotoxicity include, without limitation, multiple myeloma, mantle cell lymphoma, leukemia, digestive tract cancers, lung cancers, testicular cancers, ovarian cancers, brain cancers, uterine cancers, prostate cancers, bone cancers, breast cancers, and bladder cancers. In some cases, a mammal (e.g., a human) having multiple myeloma (e.g., relapsed multiple myeloma) and having, or at risk of developing, chemotherapy-induced (e.g., BTZ-induced) neurotoxicity can be treated by administering one or more T-type calcium channel modulators to the mammal.

In some cases, methods for treating a mammal (e.g., a human) having, or at risk of developing, neurotoxicity (e.g., chemotherapy-induced neurotoxicity), also can include identifying a mammal as having, or as being at risk of developing, neurotoxicity. Any appropriate method can be used to identify a mammal as having, or as being at risk of developing, neurotoxicity. For example, neurological examinations (e.g., neurological examinations for muscle strength, coordination, sensation, cognitive functions such as memory and thinking, and vision and speech), neurological imaging (e.g., magnetic resonance imaging (MRI)), nerve or skin biopsy, and/or electromyography (e.g., nerve conduction velocity) can be used to identify a mammal as having neurotoxicity. For example, current administration of a chemotherapeutic agent that can cause chemotherapy-induced neurotoxicity when administered to a mammal (e.g., BTZ), scheduled administration of a chemotherapeutic agent that can cause chemotherapy-induced neurotoxicity when administered to a mammal (e.g., BTZ), age (e.g., older patients are at higher risk), viral infections (e.g., herpes), history of smoking, paraneoplastic antibodies, impaired renal function with reduced creatinine clearance, pre-existing neuropathic symptoms (e.g., due to diabetes mellitus, hereditary neuropathies, and/or previous exposure to neurotoxins can be used to identify a mammal as being at risk of developing neurotoxicity (e.g., as being susceptible to developing chemotherapy-induced neurotoxicity).

Once identified as having, or as being at risk of developing, neurotoxicity (e.g., chemotherapy-induced neurotoxicity), a mammal (e.g., a human) can be administered, or instructed to self-administer, one or more T-type calcium channel modulators.

A T-type calcium channel modulator can be any molecule (e.g., a small molecule, a nucleic acid, a polypeptide, or a combination thereof) that can inhibit a T-type calcium channel. T-type calcium channels can also be referred to as voltage-activated calcium 3 (Cav3) channels. In some cases, a T-type calcium channel modulator can be a T-type calcium channel antagonist. For example, a T-type calcium channel modulator can inhibit (e.g., reduce or eliminate) expression of a T-type calcium channel (e.g., of a subunit of a T-type calcium channel). In some cases, a T-type calcium channel modulator can inhibit (e.g., can reduce or eliminate) activity of a T-type calcium channel (e.g., through binding to, or otherwise inhibiting or blocking activity of the channel). As used herein, the term “CX-8998” can also refer to CX-8998 structural analogs of CX-8998 provided that the structural analog maintains the pharmaceutical function of CX-8998 as described herein (e.g., dose-dependent tremor reduction, reduction and/or elimination of seizures, and/or reduction and/or elimination of pain). Similarly, a metabolite of CX-8998 can also refer to structural analogs of a metabolite CX-8998 provided that the structural analog maintains the pharmaceutical function of a metabolite of CX-8998 as described herein. In some cases, when a T-type calcium channel modulator is CX-8998, the CX-8998 can be metabolized into (e.g., metabolized by a mammal following administration of the T-type calcium channel modulator to the mammal) one or more metabolites of metabolites of CX-8998. Chemical names for CX-8998 include, without limitation, (R)-2-(4-Isopropylphenyl)-N-(1-(5-(2,2,2-Trifluoroethoxy)pyridin-2-yl)ethyl)acetamide and 2-(4-Isopropylphenyl)-N-{1R)-1-(5-(2,2,2-trifluoroethoxy)pyridine-2-yl)ethyl}acetamide hydrochloride. Exemplary T-type calcium channel modulators of the invention include, without limitation, CX-8998 (also referred to as MK-8998), metabolites of CX-8998, CX-5395, and CX-6526. In some cases, a mammal (e.g., a human) having, or at risk of developing, neurotoxicity can be treated by administering CX-8998 to the mammal. The chemical structure of CX-8998 is as shown below.

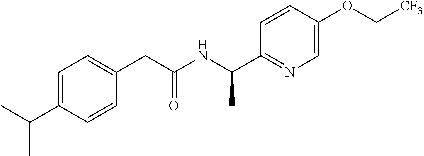 [SMILES: CC(C)c1ccc(CC(=O)N[C@H](C)c2ccc(OCC(F)(F)F)cn2)cc1]

The chemical structures of exemplary CX-8998 metabolites are as shown below.

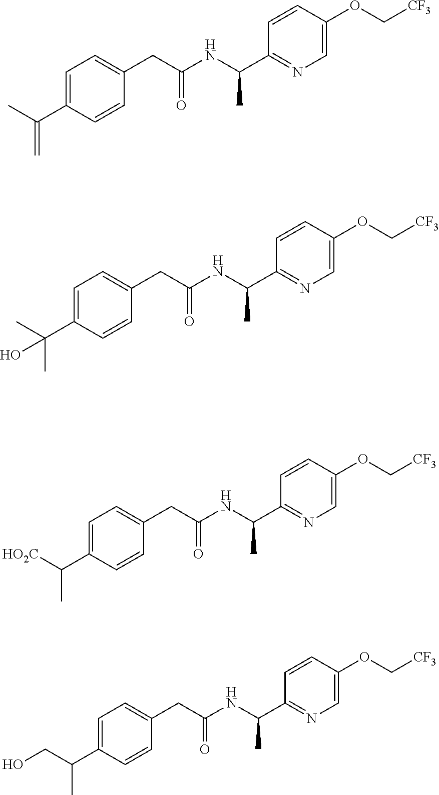 [SMILES: C=C(C)c1ccc(CC(=O)N[C@H](C)c2ccc(OCC(F)(F)F)cn2)cc1 ; C[C@@H](NC(=O)Cc1ccc(C(C)(C)O)cc1)c1ccc(OCC(F)(F)F)cn1 ; CC(C(=O)O)c1ccc(CC(=O)N[C@H](C)c2ccc(OCC(F)(F)F)cn2)cc1 ; CC(CO)c1ccc(CC(=O)N[C@H](C)c2ccc(OCC(F)(F)F)cn2)cc1]

A T-type calcium channel modulator (e.g., CX-8998 or a metabolite thereof) can be in any appropriate form. In some cases, a T-type calcium channel modulator can be in the form of a base (e.g., a free base form of the compound). In some cases, a T-type calcium channel modulator can be in the form of a salt (e.g., a salt form of the compound). In cases where a T-type calcium channel modulator such as CX-8998 or a metabolite thereof is a salt, the salt can be any appropriate salt. For example, a CX-8998 salt can include a salt formed with any appropriate acid (e.g., hydrochloric acids, citric acids, hydrobromic acids, maleic acids, phosphoric acids, sulfuric acids, fumaric acids, and tartaric acids). For example, CX-8998 can be a CX-8998 hydrochloride salt (e.g., CX-8998-HCl). In some cases, a CX-8998 salt can be deuterated.

In some cases, a T-type calcium channel modulator (e.g., CX-8998 or a metabolite thereof) can be as described elsewhere (see, e.g., International Patent Application entitled “Treating Essential Tremor Using (R)-2-(4-Isopropylphenyl)-N-(1-(5-(2,2,2-Trifluoroethoxy)pyridin-2-yl)ethyl)acetamide,” filed on Oct. 3, 2019).

In some cases, a T-type calcium channel modulator (e.g., CX-8998 or a metabolite thereof) can cross the blood brain barrier. For example, CX-8998 or a metabolite thereof can cross the blood brain barrier (e.g., can be present in the cerebrospinal fluid (CSF) and/or the CNS). In some cases, a T-type calcium channel modulator cannot cross the blood brain barrier.

A T-type calcium channel modulator (e.g., CX-8998 or a metabolite thereof) can be a selective modulator. “Selective” in this context means that the T-type calcium channel modulator is more potent at modulating T-type calcium channels compared with other voltage activated calcium channels. For example, a T-type calcium channel modulator can be more potent at modulating T-type calcium channels compared with other types of calcium channels (e.g., L-type calcium channels, P-type calcium channels, N-type calcium channels, and R-type calcium channels). For example, a T-type calcium channel modulator can be more potent at modulating T-type calcium channels compared with other types of ion channel targets (e.g., chloride channels, potassium channels, and sodium channels). Selectivity can be determined using any appropriate method. For example, selectivity can be determined by comparing the IC₅₀ of a T-type calcium modulator in inhibiting a first type of ion channel (e.g., a T-type calcium channel) with its IC₅₀ in inhibiting a second type of ion channel (e.g., a sodium channel). If the IC₅₀ for inhibiting the first type of channel is lower than the IC₅₀ for inhibiting the second type of channel, then the T-type calcium modulator can be considered selective. An IC₅₀ ratio of 0.1 (or lower) denotes 10-fold (or greater) selectivity. An IC₅₀ ratio of 0.01 (or lower) denotes 100-fold (or greater) selectivity. An IC₅₀ ratio of 0.001 (or lower) denotes 1000-fold (or greater) selectivity. In some cases, a T-type calcium channel modulator such as CX-8998 or a metabolite thereof can have selectivity for the T-type calcium that is 2-fold or greater, 10-fold or greater, 100-fold or greater, or 1000-fold or greater compared with other types of ion channels. For example, a T-type calcium channel modulator such as CX-8998 or a metabolite thereof can have greater than 100-fold selectivity over other ion channels. In some cases, a T-type calcium channel modulator such as CX-8998 or a metabolite thereof can selectively antagonize any of the Cav3 isoforms (e.g., Cav3.1, Cav3.2, and/or Cav3.3). In some cases, a T-type calcium channel modulator such as CX-8998 or a metabolite thereof can selectively antagonize all three Cav3 isoforms (e.g., Cav3.1, Cav3.2, and Cav3.3).

In some cases, one or more (e.g., one, two, three, four, five or more) T-type calcium channel modulators (e.g., CX-8998 or a metabolite thereof) can be formulated into a composition (e.g., a pharmaceutically acceptable composition) for administration to a mammal (e.g., a human) having, or at risk of developing, neurotoxicity (e.g., chemotherapy-induced neurotoxicity such as BTZ-induced neurotoxicity). For example, one or more T-type calcium channel modulators can be formulated together with one or more pharmaceutically acceptable carriers (additives), excipients, and/or diluents. In some cases, a pharmaceutically acceptable carrier, excipient, and/or diluent can be a non-naturally occurring pharmaceutically acceptable carrier, excipient, and/or diluent. In some cases, a pharmaceutically acceptable carrier, excipient, and/or diluent can be a synthetic pharmaceutically acceptable carrier, excipient, and/or diluent. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

A composition including one or more (e.g., one, two, three, four, five or more) T-type calcium channel modulators (e.g., CX-8998 or a metabolite thereof) can be designed for any type of administration to a mammal (e.g., a human) having, or at risk of developing, neurotoxicity (e.g., chemotherapy-induced neurotoxicity such as BTZ-induced neurotoxicity). For example, a composition including one or more T-type calcium channel modulators can be designed for oral or parenteral (including, without limitation, a subcutaneous, intramuscular, intravenous, intradermal, intra-cerebral, intrathecal, or intraperitoneal (i.p.) injection) administration to a mammal having, or at risk of developing, neurotoxicity. Compositions suitable for oral administration include, without limitation, liquids, tablets, capsules, pills, powders, gels, and granules. In some cases, a composition including CX-8998 or a metabolite thereof can be an immediate release oral dosage form. In some cases, a composition including CX-8998 or a metabolite thereof can be a controlled (e.g., delayed and/or sustained) release oral dosage form. In some cases, a composition including CX-8998 or a metabolite thereof can be an oral dosage form having at least a first component designed for immediate release and a second component designed for controlled release. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition including one or more (e.g., one, two, three, four, five or more) T-type calcium channel modulators (e.g., CX-8998 or a metabolite thereof) can be administered to a mammal (e.g., a human) having, or at risk of developing, neurotoxicity (e.g., chemotherapy-induced neurotoxicity such as BTZ-induced neurotoxicity) in any appropriate amount (e.g., any appropriate dose). For example, a composition described herein can be formulated to deliver an effective amount of one or more T-type calcium channel modulators to a mammal having, or at risk of developing, neurotoxicity. Effective amounts can vary depending on the route of administration, the age and general health condition of the subject, excipient usage, the possibility of co-usage with other therapeutic treatments such as use of other agents, and the judgment of the treating physician. An effective amount of a composition containing one or more T-type calcium channel modulators can be any amount that can treat a mammal having, or at risk of developing, neurotoxicity as described herein without producing significant toxicity (e.g., damage to cells (cytotoxicity), tissues, and/or organs (such as hepatotoxicity) other than nervous tissue) to the mammal. For example, an effective amount of CX-8998 can be from about 10 nM to about 1000 nM (e.g., from about 10 nM to about 900 nM, from about 10 nM to about 800 nM, from about 10 nM to about 700 nM, from about 10 nM to about 600 nM, from about 10 nM to about 500 nM, from about 10 nM to about 400 nM, from about 10 nM to about 300 nM, from about 10 nM to about 200 nM, from about 10 nM to about 100 nM, from about 10 nM to about 50 nM, from about 50 nM to about 1000 nM, from about 10 nM to about 1000 nM, from about 10 nM to about 1000 nM, from about 100 nM to about 1000 nM, from about 200 nM to about 1000 nM, from about 300 nM to about 1000 nM, from about 400 nM to about 1000 nM, from about 500 nM to about 1000 nM, from about 600 nM to about 1000 nM, from about 700 nM to about 1000 nM, from about 800 nM to about 1000 nM, from about 900 nM to about 1000 nM, from about 100 nM to about 900 nM, from about 200 nM to about 800 nM, from about 300 nM to about 700 nM, from about 400 nM to about 600 nM, from about 100 nM to about 300 nM, from about 300 nM to about 500 nM, from about 500 nM to about 700 nM, or from about 700 nM to about 900 nM). In some cases, an effective amount of CX-8998 can be about 10 nM, about 30 nM, about 100 nM, about 300 nM, or about 1000 nM. For example, an effective amount of CX-8998 can be from about 10 micrograms per kg body weight of the mammal being treated (μg/kg) to about 1000 μg/kg per day (e.g., from about 10 μg/kg to about 900 μg/kg, from about 10 μg/kg to about 800 μg/kg, from about 10 μg/kg to about 700 μg/kg, from about 10 μg/kg to about 600 μg/kg, from about 10 μg/kg to about 500 μg/kg, from about 10 μg/kg to about 400 μg/kg, from about 10 μg/kg to about 300 μg/kg, from about 10 μg/kg to about 200 μg/kg, from about 10 μg/kg to about 100 μg/kg, from about 10 μg/kg to about 50 μg/kg, from about 50 μg/kg to about 1000 μg/kg, from about 100 μg/kg to about 1000 μg/kg, from about 200 μg/kg to about 1000 μg/kg, from about 300 μg/kg to about 1000 μg/kg, from about 400 μg/kg to about 1000 μg/kg, from about 500 μg/kg to about 1000 μg/kg, from about 600 μg/kg to about 1000 μg/kg, from about 700 μg/kg to about 1000 μg/kg, from about 800 μg/kg to about 1000 μg/kg, from about 900 μg/kg to about 1000 μg/kg, from about 50 μg/kg to about 900 μg/kg, from about 75 μg/kg to about 800 μg/kg, from about 100 μg/kg to about 600 μg/kg, from about 200 μg/kg to about 700 μg/kg, from about 300 μg/kg to about 600 μg/kg, from about 400 μg/kg to about 500 μg/kg, from about 100 μg/kg to about 200 μg/kg, from about 200 μg/kg to about 300 μg/kg, from about 300 μg/kg to about 400 μg/kg, from about 400 μg/kg to about 500 μg/kg, from about 500 μg/kg to about 600 μg/kg, from about 600 μg/kg to about 700 μg/kg, from about 700 μg/kg to about 800 μg/kg, or from about 800 μg/kg to about 900 μg/kg of body weight per day). In some cases, an effective amount of CX-8998 can be about 100 μg/kg, about 300 μg/kg, or about 600 μg/kg per day. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the neurotoxicity in the mammal being treated may require an increase or decrease in the actual effective amount administered.

A composition containing one or more (e.g., one, two, three, four, five or more) T-type calcium channel modulators (e.g., CX-8998 or a metabolite thereof) can be administered to a mammal (e.g., a human) having, or at risk of developing, neurotoxicity (e.g., chemotherapy-induced neurotoxicity such as BTZ-induced neurotoxicity) in any appropriate frequency. The frequency of administration can be any frequency that can treat a mammal having, or at risk of developing, neurotoxicity without producing significant toxicity (e.g., damage to cells (cytotoxicity), tissues, and/or organs (such as hepatotoxicity) other than nervous tissue) to the mammal. For example, the frequency of administration can be from about multiple times a day (e.g., BID) to about once a day, from about once a day to about once a week, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

A composition containing one or more (e.g., one, two, three, four, five or more) T-type calcium channel modulators (e.g., CX-8998 or a metabolite thereof) can be administered to a mammal (e.g., a human) having, or at risk of developing, neurotoxicity (e.g., chemotherapy-induced neurotoxicity such as BTZ-induced neurotoxicity) for any appropriate duration. An effective duration for administering or using a composition containing one or more T-type calcium channel modulators can be any duration that can treat a mammal having, or at risk of developing, a neurotoxicity without producing significant toxicity (e.g., damage to cells (cytotoxicity), tissues, and/or organs (such as hepatotoxicity) other than nervous tissue) to the mammal. For example, the effective duration can vary from several days to several weeks, several weeks to several months, from several months to several years, or from several years to a lifetime. In some cases, the effective duration can range in duration from about 10 years to about a lifetime. Multiple factors can influence the actual effective duration used for a particular treatment. For example, an effective duration can vary with the frequency of administration, effective amount, use of multiple treatment agents, and/or route of administration.

In some cases, a composition containing one or more (e.g., one, two, three, four, five or more) T-type calcium channel modulators (e.g., CX-8998 or a metabolite thereof) can include the one or more T-type calcium channel modulator(s) as the sole active ingredient(s) in the composition effective to treat a mammal (e.g., a human) having, or at risk of developing, neurotoxicity (e.g., chemotherapy-induced neurotoxicity such as BTZ-induced neurotoxicity).

In some cases, a composition containing one or more (e.g., one, two, three, four, five or more) T-type calcium channel modulators (e.g., CX-8998 or a metabolite thereof) can include one or more (e.g., one, two, three, four, five or more) additional active agents (e.g., therapeutic agents) in the composition that are effective to treat a mammal (e.g., a human) having, or at risk of developing, neurotoxicity (e.g., chemotherapy-induced neurotoxicity such as BTZ-induced neurotoxicity).

In some cases, a mammal (e.g., a human) having, or at risk of developing, neurotoxicity (e.g., chemotherapy-induced neurotoxicity such as BTZ-induced neurotoxicity) being treated as described herein by administering one or more T-type calcium channel modulators such as CX-8998 or a metabolite thereof also can be treated with one or more (e.g., one, two, three, four, five or more) additional therapeutic agents. A therapeutic agent used in combination with one or more T-type calcium channel modulators described herein can be any appropriate therapeutic agent. In some cases, a therapeutic agent used to a treat neurotoxicity can be an agent that can reduce or eliminate one or more symptoms of neurotoxicity. Examples of therapeutic agents that can be used in combination with one or more T-type calcium channel modulators described herein to treat a mammal having, or at risk of developing, neurotoxicity include, without limitation, steroids (e.g., corticosteroids), pain medications (e.g., acetaminophen, nonsteroidal anti-inflammatory drugs (NSAIDs) such as ibuprofen and naproxen, and opioids such as hydrocodone, hydromorphone, methadone, morphine, and oxycodone), antiepileptic agents, and antidepressants (e.g., serotonin-norepinephrine reuptake inhibitors (SNRIs), selective serotonin reuptake inhibitors (SSRIs), tricyclics, and monoamine oxidase inhibitors (MAOIs)). In some cases, the one or more additional therapeutic agents can be administered together with the one or more T-type calcium channel modulators (e.g., in a composition containing one or more T-type calcium channel modulators and containing one or more additional therapeutic agents). In some cases, the one or more additional therapeutic agents can be administered independent of the one or more T-type calcium channel modulators. When the one or more additional therapeutic agents are administered independent of the one or more T-type calcium channel modulators, the one or more T-type calcium channel modulators can be administered first, and the one or more additional therapeutic agents administered second, or vice versa.

In some cases, methods for treating a mammal (e.g., a human) having, or at risk of developing, neurotoxicity (e.g., chemotherapy-induced neurotoxicity such as BTZ-induced neurotoxicity) as described herein (e.g., by administering one or more T-type calcium channel modulators such as CX-8998 or a metabolite thereof) also can include subjecting the mammal to one or more (e.g., one, two, three, four, five or more) additional treatments (e.g., therapeutic interventions) that are effective to treat a neurotoxicity to treat the mammal. Examples of additional treatments that can be used as described herein to treat a mammal having, or at risk of developing, neurotoxicity include, without limitation, oxygen therapy (e.g., hyperbaric oxygen therapy), occupational therapy, physical therapy, surgery, and meditation. In some cases, the one or more additional treatments that are effective to treat one or more symptoms of a neurotoxicity can be performed at the same time as the administration of the one or more T-type calcium channel modulators. In some cases, the one or more additional treatments that are effective to treat one or more symptoms of a neurotoxicity can be performed before and/or after the administration of the one or more T-type calcium channel modulators.

In some cases, a mammal (e.g., a human) having, or at risk of developing, neurotoxicity (e.g., chemotherapy-induced neurotoxicity such as BTZ-induced neurotoxicity) being treated as described herein by administering one or more (e.g., one, two, three, four, five or more) T-type calcium channel modulators such as CX-8998 or a metabolite thereof can be administered, or can be scheduled for administration of, one or more (e.g., one, two, three, four, five or more) chemotherapeutic agents that can cause chemotherapy-induced neurotoxicity when administered to a mammal. A chemotherapeutic agent that can cause chemotherapy-induced neurotoxicity when administered to a mammal that can be used in combination with one or more T-type calcium channel modulators described herein can be any appropriate chemotherapeutic agent that can cause chemotherapy-induced neurotoxicity when administered to a mammal. Examples of chemotherapeutic agents that can cause chemotherapy-induced neurotoxicity when administered to a mammal include, without limitation, proteasome inhibitors (e.g., BTZs such as VELCADE®, CHEMOBORT™, and BORTECAD™), epothilones, vinca alkaloids, taxanes, immunomodulatory drugs, anthracyclines, cyclophosphamides, and platinum-based therapies. For example, a mammal having cancer and being administered, or being scheduled for administration of, one or more chemotherapeutic agents that can cause chemotherapy-induced neurotoxicity when administered to a mammal also can be administered (e.g., can be co-treated with) one or more T-type calcium channel modulators. As used herein, a co-treatment or co-administration can include administration of two or more therapeutic agents (e.g., one or more T-type calcium channel modulators and one or more chemotherapeutic agents that can cause chemotherapy-induced neurotoxicity when administered to a mammal) during the course of a treatment. In some cases, co-administration of two or more therapeutic agents can include simultaneous or substantially simultaneous administration of the two or more therapeutic agents. For example, one or more T-type calcium channel modulators can be administered within seconds or minutes (e.g., from about 0 minutes to about 5 minutes apart) of the administration of one or more chemotherapeutic agents that can cause chemotherapy-induced neurotoxicity when administered to a mammal. In some cases, the one or more chemotherapeutic agents that can cause chemotherapy-induced neurotoxicity when administered to a mammal can be administered together with the one or more T-type calcium channel modulators (e.g., in a composition containing one or more T-type calcium channel modulators and containing one or more chemotherapeutic agents that can cause chemotherapy-induced neurotoxicity when administered to a mammal). In some cases, the one or more chemotherapeutic agents that can cause chemotherapy-induced neurotoxicity when administered to a mammal can be administered independent of the one or more T-type calcium channel modulators. For example, one or more T-type calcium channel modulators can be administered within minutes, hours, days, or weeks of the administration of one or more chemotherapeutic agents that can cause chemotherapy-induced neurotoxicity when administered to a mammal. When the one or more chemotherapeutic agents that can cause chemotherapy-induced neurotoxicity when administered to a mammal are administered independent of the one or more T-type calcium channel modulators, the one or more T-type calcium channel modulators can be administered first and the one or more chemotherapeutic agents that can cause chemotherapy-induced neurotoxicity when administered to a mammal administered second (e.g., the one or more T-type calcium channel modulators can be administered prophylactically), or vice versa.

In some cases, methods for treating a mammal (e.g., a human) having, or at risk of developing, neurotoxicity (e.g., chemotherapy-induced neurotoxicity such as BTZ-induced neurotoxicity) as described herein (e.g., by administering one or more T-type calcium channel modulators such as CX-8998 or a metabolite thereof) also can include monitoring the mammal being treated. Any appropriate method can be used to monitor the severity of a neurotoxicity in a mammal. For example, neurological examinations (e.g., neurological examinations for muscle strength, coordination, sensation, cognitive functions such as memory and thinking, and vision and speech), neurological imaging (e.g., MRI), nerve or skin biopsy, and/or electromyography (e.g., nerve conduction velocity) can be used to monitor the severity of a neurotoxicity in a mammal. In some cases, methods described herein also can include monitoring a mammal being treated as described herein for other types of toxicity (e.g., damage to cells (cytotoxicity), tissues, and/or organs (such as hepatotoxicity and nephrotoxicity) other than nervous tissue). The level of toxicity, if any, can be determined by assessing a mammal's clinical signs and symptoms before and after administering a known amount of a particular composition. It is noted that the effective amount of a particular composition administered to a mammal can be adjusted according to a desired outcome as well as the mammal's response and level of toxicity.

The invention will be further described in the following examples, which do not limit the scope of the invention described in the claims.

### EXAMPLES

### Example 1: Reversal of Bortezomib-Induced Neurotoxicity by CX-8998

This Example evaluates CX-8998, a selective T-type calcium channel modulator, for interference with bortezomib (BTZ) cytotoxicity and reversal of chemotherapy-induced peripheral neurotoxicity (CIPN).

### Methods

### In Vitro Studies

### Chemicals and Drugs

RPMI (Roswell Park Memorial Institute) 1640 medium, penicillin (100 U/mL), streptomycin (100 μg/mL), HEPES (4-(2-hydroxyethyl)-1-piperazineethanesulfonic acid), sodium bicarbonate and sodium pyruvate were purchased from EuroClone SpA (Pero, Italy). Fetal bovine serum (FBS) was procured from Hyclone Laboratories, Inc (Logan, Utah, USA). All other chemicals were obtained from Sigma-Aldrich (St. Louis, Mo., USA). BTZ was acquired from LC Laboratories (Woburn, Mass., USA) and CX-8998 was provided by Cavion, Inc. (Charlottesville, Va., USA). BTZ (2.6 mM) and CX-8998 (10 mM) were dissolved in dimethyl sulfoxide (DMSO) and diluted in culture medium.

### Human Multiple Myeloma Cell Lines

MM.1S and U266B1 cells were obtained from the American Type Culture Collection (San Giovanni, Italy). All cell lines were maintained in floating culture with RPMI medium that contained 2 mM L-glutamine supplemented with 10% FBS, penicillin and streptomycin. MM.1S cell medium was supplemented with 1.5 g/L sodium bicarbonate, 10 mM HEPES and 1 mM sodium pyruvate. Cells were grown in 75 cm² culture flasks for floating cells (Corning Inc. Corning, N.Y., USA) at 37° C. in 5% CO₂ and 95% air.

### In Vitro BTZ Cytotoxicity Study

The Sulfrodamide B Assay (SRB) measured cell growth inhibitory effect (% cell survival) of BTZ. Cells were plated in 96-well plates (Eppendorf, Milano, Italy) at 10000 cells/well. After 24 hours, cells were exposed to BTZ (0.05-250 nM) for 72 hours. After incubation, BTZ was diluted in culture medium at dose range for testing. Cells were fixed with trichloroacetic acid for 1 hour. Cells were stained with a solution of SRB in 1% acetic acid for 15 minutes. Unbound dye was removed by 5 washes with 1% acetic acid. Bound dye was dissolved with a solution of Tris (hydroxymethyl) aminomethane base and absorbance of content was measured at 540 nM. Growth inhibition was expressed as percentage of DMSO control absorbance of cells and corrected for absorbance before addition of drug. The 50% inhibitory concentration (IC₅₀) of % cell survival by BTZ versus control was calculated by nonlinear least squares curve fitting with GraphPad Prism software (version 4.0, GraphPad Software, Inc., La Jolla, Calif., USA).

### In Vitro Combination (BTZ and CX-8998) Interference Study

Three human MMCs (RPMI 8226, MM.1S, U266B1) were exposed for 72 hours to IC₅₀ concentration of BTZ alone or in combination with 5 concentrations (10, 30, 100, 300, 1000 nM) of CX-8998. These concentrations (over 2 orders of magnitude) of CX-8998 were based on previous cell culture experiments. Incubations with 5 concentrations of CX-8998 alone and DMSO (control) alone were also performed. Growth inhibition of each of 3 cell lines was measured by SRB assay and expressed as mean±SD % cell survival for each concentration of drug or drug combination versus control (control value of 100%). Differences in percent cell survival between each drug or drug combination and control were analyzed by non-linear least squares with statistical significance at p<0.05.

### In Vivo Studies

### In Vivo BTZ and Combination (BTZ and CX-8998) Anti-Tumor (Interference) Study

Care and husbandry of mice complied with USDA (U.S. Department of Agriculture) Animal Welfare Act and START IACUC (Institutional Animal Care and Use Committee) regulations. The protocol was reviewed and approved by START IACUC. Mice were individually housed in Sealsafe® Plus ventilated cages (Techniplast, West Chester, Pa., USA) and fed with Teklad 2919 (Envigo, Somerset, N.J., USA), irradiated, 19% protein, 9% fat and 4% fiber mouse chow. Mice were maintained under controlled environmental conditions (22+/−2° C. temperature, 55+/−10% relative humidity and 12-hour light/dark cycle (7 a.m.-7 p.m.).

South Texas Accelerated Research Therapeutics (START) (San Antonio, Tex., USA) conducted this study. BTZ and combination of BTZ and CX-8998 were tested for anti-tumor activity in a START cell-based xenograft (START-CBX) athymic nude mouse (Cr1: NU(NCr)-Foxn1^{nu} tumor model of human myeloma. RPMI-8226 cells were subcutaneously injected (10⁵ cells) into 32 female athymic nude mice (Charles River Laboratories, Houston, Tex., USA) at 6-12 weeks of age. The study was initiated when tumor volume (TV) reached 125-250 mm³. TV was estimated by measurement of palpable mass with a digital caliper and expressed in mm³ with the formula of width²×length×0.52. Mice were stratified by mean TV into 4 groups of 8 animals each: tumor vehicle control (0.5% methylene chloride and 1% Tween 80 orally once daily for 18 days), non-tumor control (no treatments for 28 days), tumor BTZ (1 mg/kg BTZ intravenous injection twice weekly for 28 days), tumor BTZ and CX-8998 (1 mg/kg BTZ intravenous injection twice weekly for 28 days and 30 mg/kg CX-8998 orally once daily for 28 days). The 30 mg/kg dose was tolerated in preclinical safety studies and anticipated to result in exposures in excess of the therapeutic range for 28 days. Body weight, TV and animal observations were collected twice per week to day 18 (termination) for vehicle control mice and twice per week to day 28 (termination) for each of the other 3 groups. Mean±SD body weight and TV were analyzed with Kruskal-Wallis and Dunn multiple comparison test at day 18 and with Mann Whitney test at day 28 with statistical significance at p<0.05.

### In Vivo BTZ and Combination (BTZ and CX-8998) CIPN-Reversal Study

Care and husbandry for the Wistar rat study conformed to University of Milano-Bicocca guidelines and complied with national (D. L. n. 26/2014) and international regulations and policies (Directive 2010/63/EU). Rats were housed 2-3 per cage under similar environmental conditions as nude mice in the interference study. The protocol (47123/14) was approved by University of Milano-Bicocca Ethics Committee.

Wistar female rats (n=52) (Envigo, Correzzano, Italy) at 10-11 weeks of age were utilized. The study was divided into 2 phases of 4 weeks each. In phase 1, rats were randomized into 2 groups: group 1 received BTZ (n=44) at 0.2 mg/kg via tail vein intravenously, 3 times per week, for 4 weeks and group 2 (n=8) was untreated (control). Body weight was measured periodically during phase 1 from baseline (day 1) to day 28. At baseline and conclusion of phase 1 (day 28), nerve conduction velocity (NCV) was performed on caudal and sciatic nerves and Dynamic Aesthesiometer Test (DAT) measured mechanical threshold (MT) of the hind paw. BTZ rats were then re-randomized into 4 groups for next phase. In phase 2, one group (n=8) remained untreated (control), second group (n=8) received BTZ at 0.2 mg/kg 3 times per week for 4 weeks and remaining 3 groups (n=12 each) received co-treatment with BTZ at 0.2 mg/kg 3 times per week for 4 weeks and CX-8998 at 3, 10, or 30 mg/kg daily by oral gavage for 4 weeks. CX-8998 doses provided a range of well tolerated exposures within and above anticipated therapeutic range based on prior preclinical studies. Body weight was measured periodically from baseline (day 28) to day 56 (termination). NCV and MT were measured in all 4 groups at baseline and on day 35 and 56. One hour after administration of BTZ, blood samples were collected for proteasome measurement on day 1, 28, 35 and 56. At termination, sciatic nerves were obtained for β-tubulin polymerization and skin samples were procured for intraepidermal nerve fiber (IENF) density and histopathology. Differences in mean±SEM body weight, mean±SEM NCV, mean±SEM MT, mean±SEM IENF density, mean±SEM β-tubulin polymerization and mean±SEM proteasome inhibition were analyzed by Mann Whitney test for comparison between CTRL and BTZ groups at the end of the 4 week treatment period, then with Kruskal-Wallis and Dunn multiple comparison test for comparison between all groups at 5 and 8 week time points with statistical significance at p<0.05.

### Assessment Methodologies for CIPN-Reversal Study

### NCV

NCV (meters/second) was obtained from caudal and sciatic nerves with an electromyography tool (Myto 2, ABN Neuro, Firenze, Italy). Caudal NCV was measured by placement of recording needle electrodes distally in the tail with stimulating needle electrodes 5 cm and 10 cm proximal to recording point. Peak latencies of potentials recorded at the 2 sites after nerve stimulation were determined and NCV was calculated. Sciatic NCV was determined by placement of needle recording electrodes near ankle bone and stimulating electrodes close to thigh. Peak latencies were recorded similar to caudal nerve and NCV was calculated. NCV was performed under standard conditions in a temperature-controlled facility (22±2° C.) and rats were under isoflurane anesthesia with monitoring of vital signs.

### DAT

MT was assessed with the DAT device (Model 37450, Ugo Basile Biological Instruments, Comerio, Italy). After acclimation, a servo-controlled pointed metallic filament (0.5 mm diameter) was placed on plantar surface of the hind paw and exerted a progressive punctate pressure up to 50 grams within 20 seconds. The pressure elicited a voluntary hind paw withdrawal response that was recorded and represented MT index. MT was collected alternatively on each side every 2 minutes on 3 occasions to yield a mean value. Mean MT values represented maximum pressure (grams) tolerated by each rat. Exposure of each animal to mechanical stimulus was limited to 30 seconds.

### Proteasome Inhibition Assay

Peripheral blood mononuclear cells (PBMC) were isolated with a Ficoll-Hypaque density separation. Cells were added to lysis solution (50 mM Hepes, 5 mM EDTA, 150 mM NaCl and Triton-X100 1% in water) and extracted. Lysates were spun at 13500 rpm for 15 minutes at 4° C. Protein extracts were solubilized in lysis buffer (10% glycerol, 25 mM TRIS-HCl pH 7.5, 1% Triton X-100, 5 mM EDTA pH8 and 1 mM EGTA pH 8) without protease and phosphate inhibitors and centrifuged at 14000 rpm for 10 minutes at 4° C. Protein concentration was assessed by Bradford assay with a Coomassie® Protein Assay Reagent Kit (Pierce, Thermo Scientific, Rockford, Ill., USA). A fluorometric assay evaluated proteasomal activity and protein extract was incubated with N-succinyl-Leu-Leu-Val-Tyr-7-Amido-4-Methylcoumarin substrate (Sigma Aldrich, Milano, Italy) for 2 hours. Proteasome activity was detected as relative light unit generated from cleaved substrate in the reagent. Fluorescence (F) from each reaction was assessed with a fluorometer (Wallac 1420 multilabel counter, Perkin Elmer Italia SPA, Monza, Italy). Proteasome activity (PA) was calculated as % PA=(F BTZ-F Substrate)/(F Control-F Substrate) and inhibition was expressed as 100×(1−PA).

### Tissue Sample Collection

At day 56, animals were euthanized by CO2 inhalation and tissue samples were procured from 4 rats per group. Right sciatic nerve was frozen in liquid nitrogen for β-tubulin polymerization assay and plantar glabrous skin samples were collected for IENF density.

β-tubulin Polymerization Assay

Protein extracts from sciatic nerves were processed similar to the proteasome assay except for lysis buffer that contained freshly added protease and phosphate inhibitors (10 mM sodium orthovanadate, 4 mM phenylmethylsulfonyl fluoride, 1% aprotinin and 20 mM sodium pyrophosphate). Protein extracts were centrifuged (14000 rpm for 10 minutes at 4° C.) to separate soluble (S) free tubulin fractions from polymerized (P) fractions. Supernatants were collected and pellets of polymerized tubulin were resuspended by sonication for 20 seconds in a volume of lysis buffer supplemented with 0.5% sodium deoxycholate (equivalent to S fraction). Protein aliquots (10 μg) were placed onto 13% SDS-PAGE and, after electrophoresis, were transferred to nitrocellulose filters. Immunoblotting analysis was performed using mouse anti-β-tubulin antibody. After incubation with primary antibody, membrane was washed and incubated with horseradish peroxidase conjugated to goat anti-rabbit IgG (Perkin Elmer Italia SPA, Monza, Italy). The ECL chemiluminescence system (Amersham GE Healthcare Europe GmbH, Milano, Italy) was used for detection. Band intensity was quantified with Gel Logic 100 Image System (Eastman Kodak, Rochester, N.Y., USA). Final mean values were obtained from triplicate experiments and data were expressed as percentage of P/P+S in treated rats compared to controls.

### IENF Density

Plantar glabrous skin samples (5 mm) from hind paws were fixed in 2% PLP (paraformaldehyde-lysine-sodium periodate) for 24 hours at 4° C. and cryoprotected overnight. Samples were serially cut with a cryostat to yield 20 μm sections. Three sections from each footpad were randomly selected and immunostained with rabbit polyclonal anti-protein gene product 9.5 (PGP 9.5; GeneTex, Irvine, Calif., USA) in combination with biotinylated anti-rabbit IgG and Vector SG substrate kit peroxidase (Vector Laboratories, Burlingame, Calif.) using a free-floating protocol. A blinded observer counted total number of immune-positive IENF in each section under light microscopy at high magnification with a microscope video camera. Individual fibers were counted that crossed dermal-epidermal interface. Secondary branches within epidermis were excluded. Length of epidermis was measured to generate linear density of IENF/millimeter as described elsewhere (Canta et al., 2016 Neurobiol Aging 45:136-148).

### Results

### In Vitro BTZ Cytotoxicity Study and In Vitro Combination (BTZ and CX-8998) Cytotoxicity Interference Study

BTZ caused concentration-dependent inhibition of cell growth (cytotoxicity) in the 3 MCLs. IC₅₀ BTZ values were 6±0.5 nM, 4±1.7 nM and 2.5±0.6 nM for MM.1S, RPMI 8226 and U266B1 cell lines, respectively.

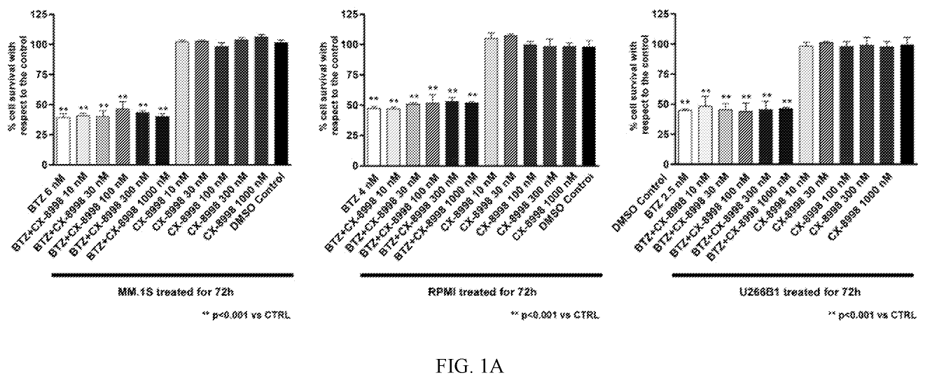

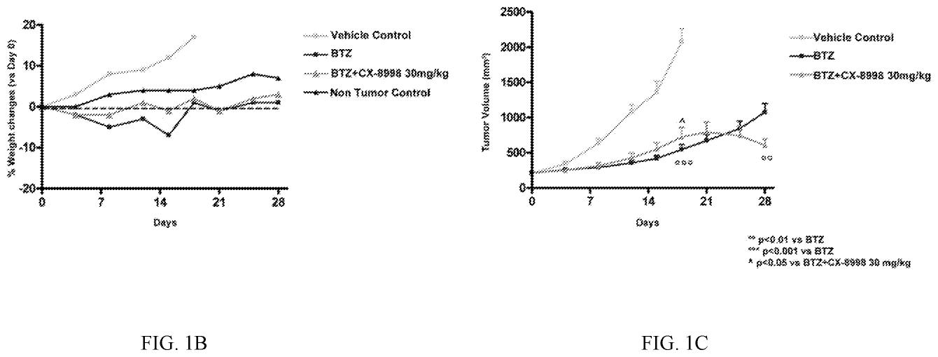

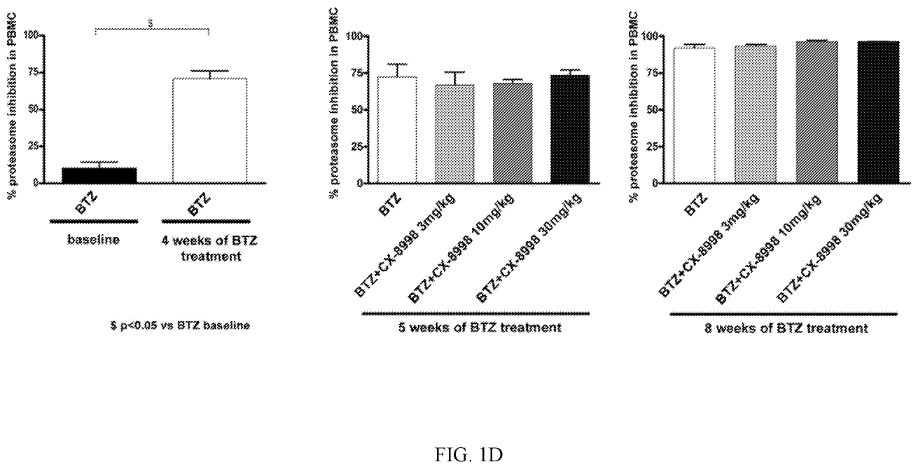

BTZ alone (IC₅₀ concentrations of 6, 4 and 2.5 nM for MM.1S, RPMI 8226 and U266B1 cell lines, respectively) significantly reduced (p<0.001) percent cell survival of the 3 MCLs compared to DMSO control (FIG. 1A). Combinations of CX-8998 (10-1000 nM) and BTZ significantly reduced (p<0.001) percent cell survival of the 3 MCLs compared to DMSO control and showed similar reduction compared to BTZ alone (FIG. 1A). CX-8998 (all concentrations) alone did not reduce percent cell survival in any of the MCLs and showed a similar level of cell survival compared to that of DMSO control (FIG. 1A).

### In Vivo Combination (BTZ and CX-8998) Anti-Tumor Interference Study

The impact of CX-8998 on BTZ anti-tumor activity in vivo was evaluated in nude mice bearing RPMI-8229 human MCL xenografts. From baseline (day 0) to day 18, percent body weight gain was evident in vehicle control group (consistent with tumor xenograft growth) and to a lesser degree in non-tumor control group (normal animal growth) (FIG. 1B). Treatment with 1 mg/kg BTZ alone or in combination with 30 mg/kg CX-8998 resulted in transient body weight loss during first two weeks of treatment and no weight gain at day 18 and 28, consistent with the known anti-tumor effect and tolerability profile of BTZ in this model (FIG. 1B).

At day 18, both BTZ alone and in combination with CX-8998 significantly reduced TV (p<0.001 and p<0.05, respectively) compared to vehicle control mice (FIG. 1C). TV tended to increase from day 18 through day 28 in mice treated with BTZ alone whereas the combination with CX-8998 tended to decrease TV and the difference between the two groups reached statistical significance (p<0.01) at termination (FIG. 1C). Overall, in vitro cell survival data and in vivo TV and weight gain data were convergent and supported lack of interference by CX-8998 with anti-tumor activity and tolerability of BTZ.

### In Vivo Combination (BTZ and CX-8998) CIPN-Reversal Study

The effects of CX-8998 on reversal of CIPN were evaluated in a rat model of BTZ induced neurotoxicity using 2-phase study design. At the end of phase 1 (week 4) there was no statistically significant difference in mean±SEM body weight (grams) between BTZ and control groups of female Wistar rats. After re-randomization and additional 1 or 4 weeks (weeks 5 and 8 respectively) of treatment, body weight change was not significantly different among BTZ, BTZ in combination with 3, 10 and 30 mg/kg of CX-8998 and control groups. Treatments with BTZ alone and in combination with CX-8998 were well tolerated. Mortality was observed in two animals, one each in combinations of BTZ with 3 mg/kg CX-8998 and 10 mg/kg CX-8998.

BTZ treatment resulted in significant inhibition of mean±SEM % proteasome activity in circulating PBMCs at week 4 compared to baseline (p<0.05) (FIG. 1D). At weeks 5 and 8, proteasome activity was similarly inhibited by BTZ and all combinations of BTZ and CX-8998 (FIG. 1D). Consistent with MCL studies in vitro and in vivo, these data suggest that co-administration of CX-8998 did not interfere with anti-proteasome activity of BTZ.

### CX-8998 Effects on Physiological and Behavioral Endpoints

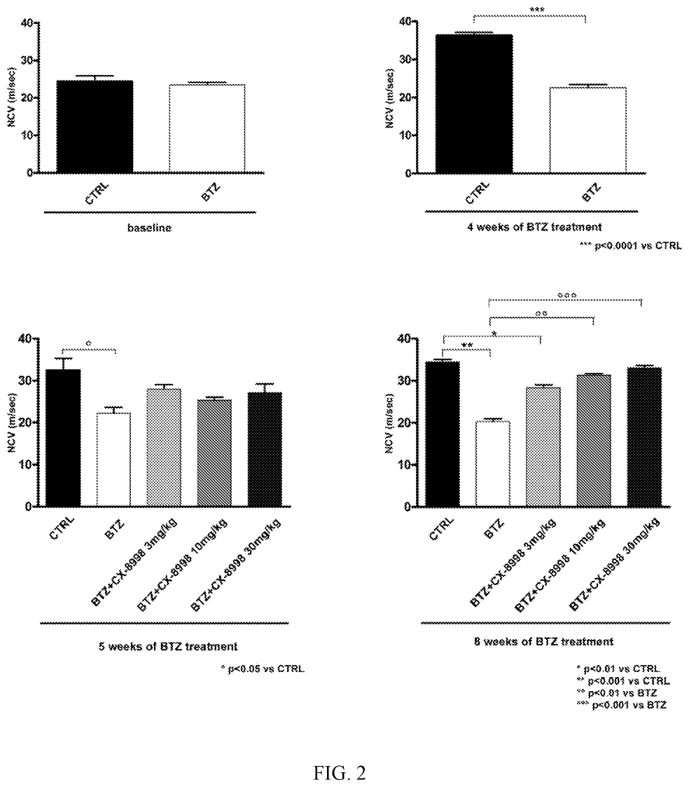

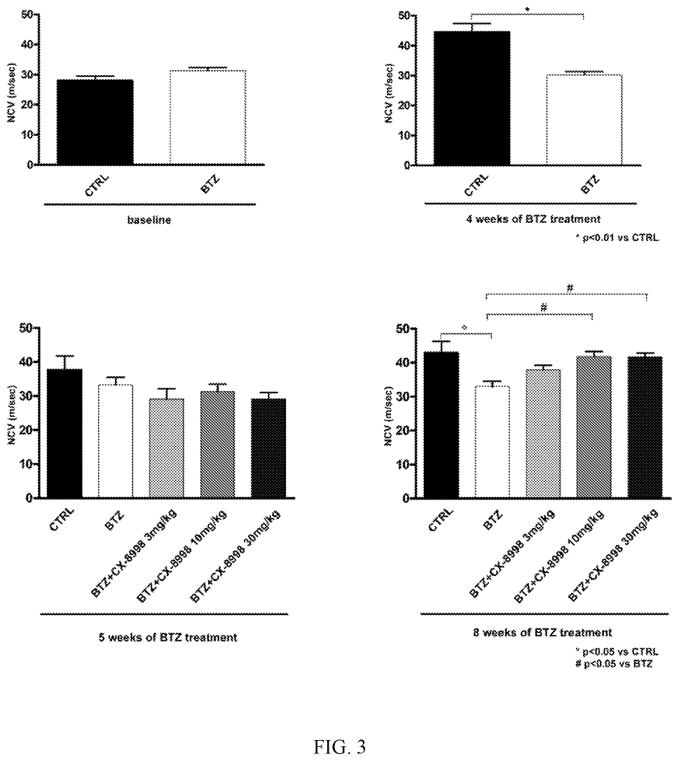

Reduced NCV and mechanical allodynia are characteristic of BTZ-induced neurotoxicity in rats (Cavaletti et al., 2007 Exp Neurol 204:317-325; and Meregalli et al., 2010 EJP 14:343-350). At baseline (day 1), mean±SEM NCV (meters/second) of caudal (FIG. 2) and sciatic (FIG. 3) nerves was similar between control and BTZ alone groups. At all post-treatment time points, caudal and sciatic NCV was significantly reduced by BTZ treatment compared to control at weeks 4, 5 and 8 for caudal nerves (p<0.01, p<0.05, p<0.001, respectively) (FIG. 2) and at weeks 4 and 8 for sciatic nerves (p<0.01, p<0.05, respectively) (FIG. 3). At week 5, NCV of sciatic nerves was numerically less than control but the difference was not statistically significant (Table 1). The greater overall variability in sciatic nerve NCV values, smaller number of animals per group at week 5 and/or lower absolute effect of BTZ on reducing NCV at week 5 versus week 8 may have contributed to lack of statistical significance.

At week 8, NCV of caudal nerves was significantly increased (p<0.01, p<0.001, respectively) by 10 and 30 mg/kg CX-8998 in combination with BTZ compared to BTZ alone (FIG. 2). NCV of sciatic nerves showed a significant increase (p<0.05) in combination groups with 10 mg/kg and 30 mg/kg CX-8998 compared to BTZ alone at week 8 (FIG. 3). At week 5, there were no significant differences in NCV of caudal or sciatic nerves in combination groups compared to BTZ alone (FIGS. 2, 3, Table 1). Caudal nerves showed a numerical trend toward increased NCV in all combinations with BTZ compared to BTZ alone at week 5 (Table 1). Conversely, a numerical trend towards decreased NCV in all combination dose groups was observed at week 5 in sciatic nerves compared to BTZ alone (Table 1). Short CX-8998 treatment duration, in addition to factors described above, may have contributed to lack of significant treatment effects observed at week 5 time point.

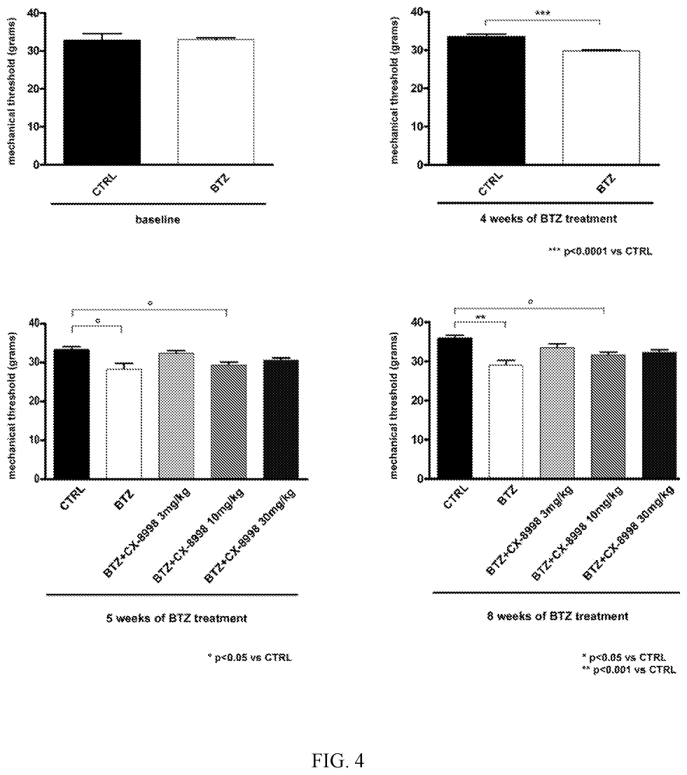

At baseline (day 1), mean±SEM MT (grams) in hind paws was comparable between control and BTZ alone groups (FIG. 4). At week 4, MT was significantly reduced (p<0.0001) by BTZ versus control rats indicating development of mechanical allodynia (FIG. 4). At weeks 5 and 8, rats treated with the combinations of BTZ and 3 and 30 mg/kg CX-8998 showed a tendency to increase MT (reduction in mechanical allodynia) compared to BTZ alone but differences were not statistically significant (FIG. 4, Table 1).

### CX-8998 Effects on Tissue Assessments

Decreased IENF density and elevated β-tubulin polymerization are tissue abnormalities associated with BTZ-induced neurotoxicity (Cavaletti et al., 2007 Exp Neurol 204:317-325; and Meregalli et al., 2010 EJP 14:343-350).

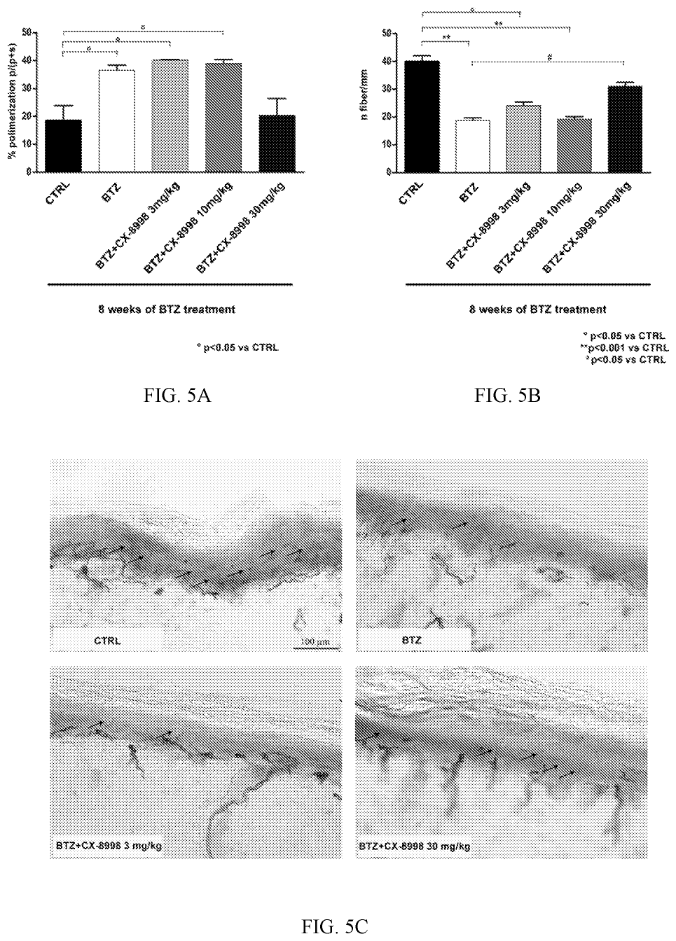

Mean±SEM β-tubulin polymerization (%) was significantly increased by BTZ alone and BTZ combined with 3 and 10 mg/kg CX-8998 compared to control (p<0.05) (FIG. 5A). This increase tended to reverse but did not reach statistical significance when BTZ was combined with 30 mg/kg CX-8998 compared to BTZ alone at week 8 (FIG. 5A, Table 1).

Mean±SEM IENF density (number of fibers per millimeter) in rat hind paw tissue samples was significantly decreased (p<0.001) by BTZ alone compared to control (FIG. 5B), consistent with impaired NCV. Co-administration of 30 mg/kg CX-8998 significantly reversed BTZ induced reduction of IENF density at week 8 (p<0.05) (FIG. 5B). Combination of BTZ and 3 mg/kg CX-8998 showed a tendency toward reversal but did not reach statistical significance compared to BTZ and no reversal was seen with co-administration of 10 mg/kg CX-8998 compared to BTZ at week 8 (FIG. 5B, Table 1).

Qualitative light microscopic analysis of nerve fibers in plantar glabrous skin of hind paw suggested that more nerve fibers (arrows) were evident with the combination of BTZ and 30 mg/kg CX-8998 compared to BTZ alone and BTZ combined with 3 mg/kg dose of CX-8998 (FIG. 5C). These qualitative nerve fiber observations are consistent with IENF density data.

TABLE 1
Treatment Group Comparisons and Statistical Analysis of Endpoints of In Vivo Studies
IENF DENSITY (FIG. 5B)

| Kruskal-Wallis test | | | |
|---|---|---|---|
| P value | P < 0.0001 | | |
| | Difference | | |
| Dunn's Multiple Comparison Test | in rank sum | P value | Summary |
| CTRL vs BTZ | 28.99 | P < 0.001 | *** |
| CTRL vs BTZ+ CX-8998 3 mg/kg | 18.06 | P < 0.05 | * |
| CTRL vs BTZ+ CX-8998 10 mg/kg | 28.94 | P < 0.001 | *** |
| CTRL vs BTZ+ CX-8998 30 mg/kg | 8.444 | P > 0.05 | ns |
| BTZ vs BTZ+ CX-8998 3 mg/kg | −10.93 | P > 0.05 | ns |
| BTZ vs BTZ+ CX-8998 10 mg/kg | −0.04167 | P > 0.05 | ns |
| BTZ vs BTZ+ CX-8998 30 mg/kg | −20.54 | P < 0.05 | * |
| BTZ+ CX-8998 3 mg/kg vs BTZ+ CX-8998 10 mg/kg | 10.89 | P > 0.05 | ns |
| BTZ+ CX-8998 3 mg/kg vs BTZ+ CX-8998 30 mg/kg | −9.611 | P > 0.05 | ns |
| BTZ+ CX-8998 10 mg/kg vs BTZ+ CX-8998 30 mg/kg | −20.50 | P < 0.01 | ** |

TUBULIN POLYMERIZATION (FIG. 5A)

| Kruskal-Wallis test | | | |
|---|---|---|---|
| P value | P < 0.0001 | | |
| | Difference | | |
| Dunn's Multiple Comparison Test | in rank sum | P value | Summary |
| CTRL vs BTZ | −14.74 | P < 0.05 | * |
| CTRL vs BTZ+ CX-8998 3 mg/kg | −15.64 | P < 0.05 | * |
| CTRL vs BTZ+ CX-8998 10 mg/kg | −15.46 | P < 0.05 | * |
| CTRL vs BTZ+ CX-8998 30 mg/kg | 0.2576 | P > 0.05 | ns |
| BTZ vs BTZ+ CX-8998 3 mg/kg | −4.000 | P > 0.05 | ns |
| BTZ vs BTZ+ CX-8998 10 mg/kg | −4.000 | P > 0.05 | ns |
| BTZ vs BTZ+ CX-8998 30 mg/kg | 15.00 | P > 0.05 | ns |
| BTZ+ CX-8998 3 mg/kg vs BTZ+ CX-8998 10 | 0.000 | P > 0.05 | ns |
| mg/kg | | | |
| BTZ+ CX-8998 3 mg/kg vs BTZ+ CX-8998 30 mg/kg | 19.00 | P < 0.05 | * |
| BTZ+ CX-8998 10 mg/kg vs BTZ+ CX-8998 30 mg/kg | 19.00 | P < 0.05 | * |

TUMOR GROWTH DAY 28 (FIG. 1B right)

| | |
|---|---|
| Mann Whitney test | |
| P value | 0.0070 |
| Exact or approximate P value? | Gaussian |
| | Approximation |
| P value summary | ** |
| Are medians signif. different? (P < 0.05) | Yes |
| One- or two-tailed P value? | Two-tailed |
| Sum of ranks in column A (BTZ), B (BTZ+ CX-8998) | 92.50, 43.50 |
| Mann-Whitney U | 7.500 |

TUMOR GROWTH DAY 18 (FIG. 1B right)

| Kruskal-Wallis test | | | |
|---|---|---|---|
| P value | P < 0.0005 | | |
| | Difference | | |
| Dunn's Multiple Comparison Test | in rank sum | P value | Summary |
| Vehicle Control vs BTZ | 13.13 | P < 0.001 | *** |
| Vehicle Control vs BTZ+ cx-8998 | 10.13 | P < 0.05 | * |
| BTZ vs BTZ+ CX-8998 | −3.000 | P > 0.05 | ns |

PROTEASOME INHIBITION baseline vs BTZ 4 weeks (FIG. 1C)

| | |
|---|---|
| Mann Whitney test | |
| P value | 0.0286 |
| Exact or approximate P value? | Exact |
| P value summary | * |
| Are medians signif. different? (P < 0.05) | Yes |
| One- or two-tailed P value? | Two-tailed |
| Sum of ranks in column A (BTZ baseline), B (BTZ 4 wks) | 10, 26 |
| Mann-Whitney U | 0.0000 |

DYNAMIC TEST BASELINE (FIG. 4)

| | |
|---|---|
| Mann Whitney test | |
| P value | 0.6572 |
| Exact or approximate P value? | Gaussian |
| | Approximation |
| P value summary | ns |
| Are medians signif. different? (P < 0.05) | No |
| One- or two-tailed P value? | Two-tailed |
| Sum of ranks in column A (CTRL), B (BTZ) | 194, 1184 |
| Mann-Whitney U | 158.0 |

DYNAMIC TEST 4 WEEKS (FIG. 4)

| | |
|---|---|
| Mann Whitney test | |
| P value | 0.0001 |
| Exact or approximate P value? | Gaussian |
| | Approximation |
| P value summary | *** |
| Are medians signif. different? (P < 0.05) | Yes |
| One- or two-tailed P value? | Two-tailed |
| Sum of ranks in column A (CTRL), B (BTZ) | 322.5, 758.5 |
| Mann-Whitney U | 17.50 |

DYNAMIC TEST 5 WEEKS (FIG. 4)

| Kruskal-Wallis test | | | |
|---|---|---|---|
| P value | P < 0.0065 | | |
| | Difference | | |
| Dunn's Multiple Comparison Test | in rank sum | P value | Summary |
| CTRL vs BTZ | 17.24 | P < 0.05 | * |
| CTRL vs BTZ+ CX-8998 3 mg/kg | 3.250 | P > 0.05 | ns |
| CTRL vs BTZ+ CX-8998 10 mg/kg | 16.50 | P < 0.05 | * |
| CTRL vs BTZ+ CX-8998 30 mg/kg | 10.56 | P > 0.05 | ns |
| BTZ vs BTZ+ CX-8998 3 mg/kg | −13.99 | P > 0.05 | ns |
| BTZ vs BTZ+ CX-8998 10 mg/kg | −0.7411 | P > 0.05 | ns |
| BTZ vs BTZ+ CX-8998 30 mg/kg | −6.679 | P > 0.05 | ns |
| BTZ+ CX-8998 3 mg/kg vs BTZ+ CX-8998 10 mg/kg | 13.25 | P > 0.05 | ns |
| BTZ+ CX-8998 3 mg/kg vs BTZ+ CX-8998 30 mg/kg | 7.313 | P > 0.05 | ns |
| BTZ+ CX-8998 10 mg/kg vs BTZ+ CX-8998 30 mg/kg | −5.938 | P > 0.05 | ns |

DYNAMIC TEST 8 WEEKS (FIG. 4)

| Kruskal-Wallis test | | | |
|---|---|---|---|
| P value | P < 0.0011 | | |
| | Difference | | |
| Dunn's Multiple Comparison Test | in rank sum | P value | Summary |
| CTRL vs BTZ | 23.19 | P < 0.001 | *** |
| CTRL vs BTZ+ CX-8998 3 mg/kg | 7.563 | P > 0.05 | ns |
| CTRL vs BTZ+ CX-8998 10 mg/kg | 16.69 | P < 0.05 | * |
| CTRL vs BTZ+ CX-8998 30 mg/kg | 13.50 | P > 0.05 | ns |
| BTZ vs BTZ+ CX-8998 3 mg/kg | −15.63 | P > 0.05 | ns |
| BTZ vs BTZ+ CX-8998 10 mg/kg | −6.500 | P > 0.05 | ns |
| BTZ vs BTZ+ CX-8998 30 mg/kg | −9.688 | P > 0.05 | ns |
| BTZ+ CX-8998 3 mg/kg vs BTZ+ CX-8998 10 mg/kg | 9.125 | P > 0.05 | ns |
| BTZ+ CX-8998 3 mg/kg vs BTZ+ CX-8998 30 mg/kg | 5.938 | P > 0.05 | ns |
| BTZ+ CX-8998 10 mg/kg vs BTZ+ CX-8998 30 mg/kg | −3.188 | P > 0.05 | ns |

NCV OF CAUDAL NERVE (BASELINE) (FIG. 2)

| | |
|---|---|
| Mann Whitney test | |
| P value | 0.4464 |
| Exact or approximate P value? | Gaussian |
| | Approximation |
| P value summary | Ns |
| Are medians signif. different? (P < 0.05) | No |
| One- or two-tailed P value? | Two-tailed |
| Sum of ranks in column A (CTRL), B (BTZ) | 187, 633 |
| Mann-Whitney U | 105.0 |

NCV OF CAUDAL NERVE (4 weeks) (FIG. 2)

| | |
|---|---|
| Mann Whitney test | |
| P value | 0.0029 |
| Exact or approximate P value? | Gaussian |
| | Approximation |
| P value summary | ** |
| Are medians signif. different? (P < 0.05) | Yes |
| One- or two-tailed P value? | Two-tailed |
| Sum of ranks in column A (CTRL), B (BTZ) | 74 , 136 |
| Mann-Whitney U | 0.0000 |

NCV OF CAUDAL NERVE (5 weeks) (FIG. 2)

| Kruskal-Wallis test | | | |
|---|---|---|---|
| P value | P < 0.0200 | | |
| | Difference | | |
| Dunn's Multiple Comparison Test | in rank sum | P value | Summary |
| CTRL vs BTZ | 15.75 | P < 0.05 | * |
| CTRL vs BTZ+ CX-8998 3 mg/kg | 4.542 | P > 0.05 | ns |
| CTRL vs BTZ+ CX-8998 10 mg/kg | 11.13 | P > 0.05 | ns |
| CTRL vs BTZ+ CX-8998 30 mg/kg | 4.708 | P > 0.05 | ns |
| BTZ vs BTZ+ CX-8998 3 mg/kg | −11.21 | P > 0.05 | ns |
| BTZ vs BTZ+ CX-8998 10 mg/kg | −4.625 | P > 0.05 | ns |
| BTZ vs BTZ+ CX-8998 30 mg/kg | −11.04 | P > 0.05 | ns |
| BTZ+ CX-8998 3 mg/kg vs BTZ+ CX-8998 10 mg/kg | 6.583 | P > 0.05 | ns |
| BTZ+ CX-8998 3 mg/kg vs BTZ+ CX-8998 30 mg/kg | 0.1667 | P > 0.05 | ns |
| BTZ+ CX-8998 10 mg/kg vs BTZ+ CX-8998 30 mg/kg | −6.417 | P > 0.05 | ns |

NCV OF CAUDAL NERVE (8 weeks) (FIG. 2)

| Kruskal-Wallis test | | | |
|---|---|---|---|
| P value | P < 0.0001 | | |
| | Difference | | |
| Dunn's Multiple Comparison Test | in rank sum | P value | Summary |
| CTRL vs BTZ | 37.00 | P < 0.001 | *** |
| CTRL vs BTZ+ CX-8998 3 mg/kg | 25.45 | P < 0.01 | ** |
| CTRL vs BTZ+ CX-8998 10 mg/kg | 14.64 | P > 0.05 | ns |
| CTRL vs BTZ+ CX-8998 30 mg/kg | 6.583 | P > 0.05 | ns |
| BTZ vs BTZ+ CX-8998 3 mg/kg | −11.55 | P > 0.05 | ns |
| BTZ vs BTZ+ CX-8998 10 mg/kg | −22.36 | P < 0.01 | ** |
| BTZ vs BTZ+ CX-8998 30 mg/kg | −30.42 | P < 0.001 | *** |
| BTZ+ CX-8998 3 mg/kg vs BTZ+ CX-8998 10 mg/kg | −10.82 | P > 0.05 | ns |
| BTZ+ CX-8998 3 mg/kg vs BTZ+ CX-8998 30 mg/kg | −18.87 | P < 0.05 | * |
| BTZ+ CX-8998 10 mg/kg vs BTZ+ CX-8998 30 mg/kg | −8.053 | P > 0.05 | ns |

NCV OF SCIATIC NERVE (BASELINE) (FIG. 3)

| | |
|---|---|
| Mann Whitney test | |
| P value | 0.1873 |
| Exact or approximate P value? | Gaussian |
| | Approximation |
| P value summary | Ns |
| Are medians signif. different? (P < 0.05) | No |
| One- or two-tailed P value? | Two-tailed |
| Sum of ranks in column A (CTRL), B (BTZ) | 124.5, 695.5 |
| Mann-Whitney U | 88.50 |

NCV OF SCIATIC NERVE (4 weeks) (FIG. 3)

| | |
|---|---|
| Mann Whitney test | |
| P value | 0.0029 |
| Exact or approximate P value? | Gaussian |
| | Approximation |
| P value summary | ** |
| Are medians signif. different? (P < 0.05) | Yes |
| One- or two-tailed P value? | Two-tailed |
| Sum of ranks in column A (CTRL), B (BTZ) | 74, 136 |
| Mann-Whitney U | 0.0000 |

NCV OF SCIATIC NERVE (5 weeks) (FIG. 3)

| Kruskal-Wallis test | | | |
|---|---|---|---|
| P value | P < 0.3513 | | |
| | Difference | | |
| Dunn's Multiple Comparison Test | in rank sum | P value | Summary |
| CTRL vs BTZ | 2.250 | P > 0.05 | ns |
| CTRL vs BTZ+ CX-8998 3 mg/kg | 8.917 | P > 0.05 | ns |
| CTRL vs BTZ+ CX-8998 10 mg/kg | 4.833 | P > 0.05 | ns |
| CTRL vs BTZ+ CX-8998 30 mg/kg | 7.500 | P > 0.05 | ns |
| BTZ vs BTZ+ CX-8998 3 mg/kg | 6.667 | P > 0.05 | ns |
| BTZ vs BTZ+ CX-8998 10 mg/kg | 2.583 | P > 0.05 | ns |
| BTZ vs BTZ+ CX-8998 30 mg/kg | 5.250 | P > 0.05 | ns |
| BTZ+ CX-8998 3 mg/kg vs BTZ+ CX-8998 10 mg/kg | −4.083 | P > 0.05 | ns |
| BTZ+ CX-8998 3 mg/kg vs BTZ+ CX-8998 30 mg/kg | −1.417 | P > 0.05 | ns |
| BTZ+ CX-8998 10 mg/kg vs BTZ+ CX-8998 30 mg/kg | 2.667 | P > 0.05 | ns |

NCV OF SCIATIC NERVE (8 weeks) (FIG. 3)

| Kruskal-Wallis test | | | |
|---|---|---|---|
| P value | P < 0.0059 | | |
| | Difference | | |
| Dunn's Multiple Comparison Test | in rank sum | P value | Summary |
| CTRL vs BTZ | 22.10 | P < 0.05 | * |
| CTRL vs BTZ+ CX-8998 3 mg/kg | 11.98 | P > 0.05 | ns |
| CTRL vs BTZ+ CX-8998 10 mg/kg | 2.848 | P > 0.05 | ns |
| CTRL vs BTZ+ CX-8998 30 mg/kg | 2.333 | P > 0.05 | ns |
| BTZ vs BTZ+ CX-8998 3 mg/kg | −10.12 | P > 0.05 | ns |
| BTZ vs BTZ+ CX-8998 10 mg/kg | −19.26 | P < 0.05 | * |
| BTZ vs BTZ+ CX-8998 30 mg/kg | −19.77 | P < 0.05 | * |
| BTZ+ CX-8998 3 mg/kg vs BTZ+ CX-8998 10 mg/kg | −9.136 | P > 0.05 | ns |
| BTZ+ CX-8998 3 mg/kg vs BTZ+ CX-8998 30 mg/kg | −9.652 | P > 0.05 | ns |
| BTZ+ CX-8998 10 mg/kg vs BTZ+ CX-8998 30 mg/kg | −0.5152 | P > 0.05 | ns |

Together these results demonstrate that CX-8998 can be used to reverse neurotoxicity in CIPN models without affecting BTZ cytotoxicity.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having neurotoxicity, wherein said method comprises administering an effective amount of a composition comprising a T-type calcium channel modulator or a salt thereof to said mammal to reduce a symptom of said neurotoxicity in said mammal.

2. The method of claim 1, wherein said method comprises identifying said mammal as having said neurotoxicity.

3. The method of any one of claims 1-2, wherein said mammal is a human.

4. The method of any one of claims 1-3, wherein neurotoxicity is a chemotherapy-induced neurotoxicity.

5. The method of claim 4, wherein said chemotherapy-induced neurotoxicity is a bortezomib-induced neurotoxicity.

6. The method of any one of claims 4-5, wherein said mammal having neurotoxicity has been administered said chemotherapy to treat a cancer within said mammal.

7. The method of claim 6, wherein said cancer is selected from the group consisting of multiple myeloma, mantle cell lymphoma, leukemia, digestive tract cancers, lung cancers, testicular cancers, ovarian cancers, brain cancers, uterine cancers, prostate cancers, bone cancers, breast cancers, and bladder cancers.

8. The method of any one of claims 1-7, wherein said symptom is selected from the group consisting of pain, limb weakness, limb numbness, itch, parasthesia, palsy, anosmia, ptosis, chronic cough, motor dysfunction, memory loss, vision loss, headache, cognitive impairment, encephalopathy, dementia, mood disorder, constipation, sexual dysfunction, bladder retention, and hemorrhage.

9. The method of any one of claims 1-8, wherein said T-type calcium channel modulator is a negative modulator.

10. The method of claim 9, wherein said negative modulator is a negative allosteric modulator.

11. The method of any one of claims 1-10, wherein said T-type calcium channel modulator reduces T-type calcium channel activity.

12. The method of any one of claims 1-11, wherein said T-type calcium channel modulator comprises CX-8998.

13. The method of claim 12, wherein said CX-8998 is in the form of a salt.

14. The method of any one of claims 1-11, wherein said T-type calcium channel modulator comprises a metabolite of CX-8998.

15. The method of claim 14, wherein said metabolite of CX-8998 is selected from the group consisting of: 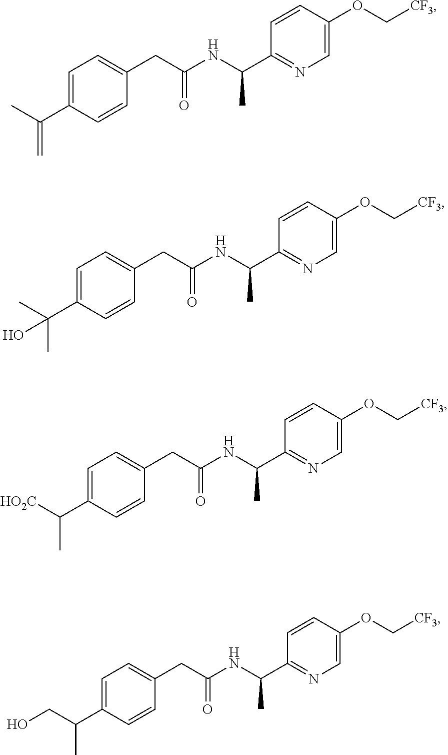 [SMILES: C=C(C)c1ccc(CC(=O)N[C@H](C)c2ccc(OCC(F)(F)F)cn2)cc1 ; C[C@@H](NC(=O)Cc1ccc(C(C)(C)O)cc1)c1ccc(OCC(F)(F)F)cn1 ; CC(C(=O)O)c1ccc(CC(=O)N[C@H](C)c2ccc(OCC(F)(F)F)cn2)cc1 ; CC(CO)c1ccc(CC(=O)N[C@H](C)c2ccc(OCC(F)(F)F)cn2)cc1]
and combinations thereof.

16. The method of claim 15 wherein said metabolite of CX-8998 is in the form of a salt.

17. The method of any one of claims 1-11, wherein said T-type calcium channel modulator comprises CX-8998 and one or more metabolites of CX-8998.

18. The method of any one of claims 1-17, wherein said composition comprises from about 10 nM to about 1000 nM of said T-type calcium channel modulator.

19. The method of any one of claims 1-18, wherein said composition comprises from about 3 mg/kg body weight of said mammal to about 30 mg/kg body weight of said mammal of said T-type calcium channel modulator to said mammal.

20. The method of any one of claims 1-19, wherein said composition is administered orally.
